(19) United States
(12) Patent Application Publication
(10) Pub. No.: US 2012/0020879 A1
(43) Pub. Date: Jan. 26, 2012

(54) PROCESS, COMPOSITION AND METHOD FOR ANION DEPOSITION INTO FERRITIN FOR THERAPEUTIC AND OTHER USE

(75) Inventor: Richard K. Watt, Provo, UT (US)

(73) Assignee: Brigham Young University, Provo, UT (US)

(21) Appl. No.: 13/189,450

(22) Filed: Jul. 22, 2011

Related U.S. Application Data
(60) Provisional application No. 61/400,194, filed on Jul. 22, 2010.

(51) Int. Cl. A61K 51/12; A61K 39/395; A61P 35/00; C06B 45/12; A61K 9/14; A61K 51/00; B82Y 5/00; B82Y 40/00

(52) U.S. Cl. 424/1.29; 424/489; 424/130.1; 424/1.11; 424/172.1; 149/14; 977/895; 977/904; 977/906

(57) ABSTRACT

Provided herein is a process for production of a metal nanoparticle, the process comprising providing a first solution containing a protein nanocage complex comprising a hydrophobic metal core and an ion-transport mechanism, providing a second solution containing a preselected anionic agent, combining the first and second solutions into a third combined solution, and applying an external method to the third combined solution to manipulate the metal core's redox state, in which reduction of the metal core causes the preselected anionic agent to be imported and incorporated into the metal core. Also provided herein is a composition from the process and a method of use.

### CROSS-REFERENCES TO RELATED APPLICATIONS

This application is a continuation-in-part of and claims priority to U.S. Provisional Patent Application No. 61/400,194 entitled “ANION DEPOSITION INTO FERRITIN” and filed on Jul. 22, 2010 for Richard Watt, which is incorporated herein by reference.

### FIELD

This invention relates to the iron storage protein ferritin and more particularly relates to the deposition of high concentrations of anions within the ferritin cage and to therapeutic use.

### BACKGROUND

In biological systems, the protein-cage ferritin is used to store iron and to keep it from building to toxic levels in cells. The structures of many ferritins isolated from a wide range of biological tissues have been determined as well as structures of recombinant proteins containing only heavy (H) and light (L) subunits. A common architecture has been conserved throughout biological species and consists of a spherical molecule composed of 24 subunits arranged in 4, 3, 2 symmetry with an outside diameter of 12 nm and a hollow internal cavity 8 nm in diameter. Six channels lined with hydrophobic residues □0.4 nm in diameter pass through the 2 nm protein shell at the 4-fold axes. Eight channels, □0.4 nm in diameter, lined with carboxylate groups are formed at the 3-fold axes. Molecular traffic passing from the surrounding exterior solution into the ferritin hollow interior is proposed to occur through the 3-fold channels. Ferritin is remarkably stable to temperature and pH changes, as evidenced by its stability up to 70° C. and over pH extremes of 3-10. At pH less than 3, the 24 subunits dissociate but reversibly reassemble at pH greater than 3. These characteristics allow for ferritin applications as an efficient drug delivery system since ferritin, have several advantages over synthetic nanoparticles, including precise dimensions, possible evasion by the immune system, biocompatibility and biodegradability.

Ferritin protein nanocages been recognized for their ability to store metal ions. The metal sequestering ability of ferritin has been used for the size-constrained synthesis of many metal nanoparticles. In addition to metal cation sequestration, ferritin is required to sequester large quantities of anions to compensate for the charge of the metal ions. The counter anion for physiological mineral formation with iron is OH−, but the core also contains ˜10% phosphate in mammalian ferritin and significantly more phosphate if the ferritin comes from bacterial sources. Other oxoanions can substitute for OH− or phosphate during iron loading in ferritin. Most of the characterized reactions with anion loading into ferritin have been performed by adding the anion at the same time as the metal is deposited into ferritin. This co-deposition process leads to simultaneous deposition of the cation and anion.

Loaded ferritin nanoparticles have been used therapeutically, with radioisotope particles sometimes targeted to specific tissues for cancer treatment, but the concentration of radioisotope is limited under current techniques, restricting the effectiveness. The traditional therapy administers a large dose of 131-1 to a patient with thyroid cancer to kill the thyroid cells. Side-effects include low sperm count and potential sterility, secondary tumors, bladder cancer and leukemia. A need exists for more highly loaded nanoparticles, which could be beneficial for a variety of scientific, industrial, commercial, and medical uses.

### SUMMARY

Provided herein is a process for production of a metal nanoparticle, the process comprising providing a first solution containing a protein nanocage complex comprising a hydrophobic metal core and an ion-transport mechanism, providing a second solution containing a preselected anionic agent, combining the first and second solutions into a third combined solution, and applying an external method to the third combined solution to manipulate the metal core's redox state. In the process provided a reduction of the metal core causes the preselected anionic agent to be imported and incorporated into the metal core.

In some embodiments the first solution is a pH-adjusted MOPS buffer. The protein nanocage complex may be selected from ferritin and ferritin-like protein complexes, which use the ion-transport mechanism to incorporate the preselected anionic agent into the metal core. The ferritin protein is sometimes an equine spleen holoferritin HoSF. In certain embodiments the holoferritin protein contains from at least 1000 iron atoms per holoferritin.

The hydrophobic metal core sometimes comprises a transition metal which may be selected from the group consisting of cobalt, iron, manganese, vanadium, nickel, zinc, copper, and silver. In certain embodiments the preselected anionic agent is selected from the group of anions consisting of a halide, a oxoanion, other anion, a radioisotope, and combinations thereof. In some embodiments the second solution comprises an anion salt of the preselected anion agent. The halide is sometimes selected from a group consisting of Fluoride (F−), Chloride (Cl−), Bromide (Br−); Iodide (I−), and combinations thereof. The oxoanion may be selected from a group comprising Nitrate (NO3−), Rhenium Trioxide (ReO3−), Phosphate (PO43−), Vandate (VO3−), Perchlorate (ClO4−); Molybdate (MoO43−); Tungstate (WO43−), and combinations thereof. In various embodiments the said other anion is selected from the group consisting of Cyanide (CN−), Thiocynate (SCN−), Azide (N3−), and combinations thereof. In certain embodiments the radioisotope is selected from the group consisting of: Molybdenum-99m (99mMo), Technetium-99m (99mTc); Lutetium-177 (177Lu); yttrium-88 (88Y); yttrium-90 (90Y), Rhenium-186 (186Rh), Rhenium-188 (188Rh), Iodine-123 (123I); Iodine-124 (124I); Iodine-125 (125I); Iodine-131 (131I); phosphorus-32 (32P), phosphorus-33 (33P); bromine-77 (77Br); and combinations thereof.

In some embodiments said third solution comprises an equimolar combination of the preselected anionic agent and the metal core. In certain embodiments the third solution is de-oxygenated. The external method is sometimes a reducing agent that creates a charge imbalance within the core and induces the importation and incorporation of the preselected anionic agent into the metal core. In various embodiments the reducing agent is formamidine sulfinic acid.

Also provided herein is a composition comprising a ferritin protein nanocage, at least 1000 molecules of a preselected anionic agent, and an antibody targeted to a specific cell. In some embodiments the compositions comprises a pharmaceutical formulation. In certain embodiments the preselected anionic agent is a radioisotope. The radioisotope is sometimes Iodine-131. In various embodiments the ferritin protein is an equine spleen holoferritin HoSF. The said antibody may be targeted to a thyroid cell.

Further provided herein is a method for at least one of diagnosing and treating a disease. In some embodiments the method provided comprises providing to a patient in need of treatment a composition comprising a ferritin nanocage, at least 1000 molecules of a radioisotope, and an antibody targeted to a specific cell.

The patient may be selected from the group consisting of mammal, bird, reptile, and amphibian. In some embodiments the disease is cancer. In certain embodiments the mammal is a human. The radioisotope is sometimes Iodine-131. In certain embodiments the disease is thyroid cancer.

Further provided herein is a composition comprising a ferritin protein nanocage, at least 1000 molecules of a preselected anionic agent, and at least 1000 molecules of a preselected cations within a metal core. In some embodiments the composition further comprises a bio-nano-propellant. The bio-nano-propellant is sometimes a rocket fuel. The rocket fuel may comprise an explosive. In certain embodiments the preselected anionic agent is Perchlorate (ClO4−). In various embodiments the preselected anionic agent is Azide (N3−). In some embodiments the preselected anionic agent is Nitrate (NO3−). The preselected cation is sometimes a divalent cation. In various embodiments the divalent cation is Iron.

Reference throughout this specification to features or similar language does not imply that all of the features that may be realized with the present invention should be or are in any single embodiment of the invention. Rather, language referring to the features is understood to mean that a specific feature or characteristic described in connection with an embodiment is included in at least one embodiment of the present invention. Thus, discussion of the features and characteristics, and similar language, throughout this specification may, but do not necessarily, refer to the same embodiment.

Furthermore, the described features, and characteristics of the invention may be combined in any suitable manner in one or more embodiments. One skilled in the relevant art will recognize that the invention can be practiced without one or more of the specific features or characteristics of a particular embodiment. In other instances, additional features and characteristics may be recognized in certain embodiments that may not be present in all embodiments of the invention.

These features and characteristics of the present invention will become more fully apparent from the following description and appended claims, or may be learned by the practice of the invention as set forth hereinafter.

### BRIEF DESCRIPTION OF THE DRAWINGS

FIG. 1 depicts a representative standard curve for ISE work. Specifically, this particular curve is for the determination of Cl− concentration. The concentration is plotted vs. the electrode readout (voltage, mV), and is shown in the inset. To obtain the standard curve, the −log [ ] is plotted vs. voltage. Also shown is the R2 value and the line equation. Sample concentrations are determined by measuring the voltage (y-value) and calculating for the −log [ ] (x-value). The concentration is determined by taking 10−log [ ].

FIG. 2 depicts a graphical schematic of the mechanism of anion entry, demonstrating halide (X−) accumulation within ferritin upon reduction and OH− ions being expelled. These reactions are reversed upon oxidation of the iron core. Two potential pathways that account for the increased hydroxide ion concentration increase on the exterior of ferritin upon reduction of the iron core. The current study focuses on the accumulation of anions in ferritin upon chemical reduction of the iron core and the release of these anions when the core is re-oxidized.

FIGS. 3A and 3B depict elution profile of samples run over a G-25 column. A) Molybdate elution profile. B) Tungstate elution profile. The black traces represent the protein elution profiles, whereas the colored traces are the oxoanions. The lighter lines are the samples prior to reduction, and the darker lines are the samples following FSA reduction.

FIG. 4 depicts spectra of the oxidized native ferritin core (black) compared to ferritin cores reduced in the presence of halides. Fe(X) OH cores (cyan, OH−; yellow, F−; green, Cl−; orange, Br−; and blue, I−). Inset depicts the solution color prior to (left) and following (right) reduction with formamidine sulfinic acid (FSA). Following oxidation, the anions are expelled, the solution turns reddish-brown, and the spectra return to the spectrum represented by the black trace.

FIG. 5 depicts scanning transmission electron micrographs of ferritin before reduction (left), and after reduction (right) of the mineral core reduced in the presence of anions (representative micrographs). The sample is not negatively stained, thus the density represents the iron core within ferritin and not the protein.

FIG. 6 depicts an EDX showing a unique spectra for the various Fe(X) OH cores, where X is a halide. Black is the control with no ferritin placed on the grid. The remaining spectra are of Fe(F)OH, yellow; Fe(Cl)OH, green; Fe(Br)OH, orange; and Fe(I) OH, blue.

FIG. 7 depicts a graphic schematic of the reductive incorporation of anions into ferritin.

FIG. 8 depicts a graphic schematic of oxidative release of anions from ferritin.

### DETAILED DESCRIPTION

Provided herein is a process, composition, and method of treatment for the production of an ion-loaded protein molecule, including but not limited to ferritin and ferritin-like proteins, and the use of said molecule in some embodiments with a cell targeting agent such as an antibody for the treatment of disease. In certain embodiments the process and composition provided is useful for additional scientific, industrial, commercial, and medical uses.

Introduction

Ferritin is the iron storage protein in cells. It is composed of 24 similar subunits that assemble to form a hollow spherical protein complex. The exterior diameter is 12 nm and the interior diameter is 8 nm. Calculations show that if the hollow interior was completely filled it could accommodate up to 4500 iron atoms. The mineral composition is ferrihydrite or FeOOH which is a dehydrated form of Fe(OH)3. The core will be designated hereafter as Fe(OH)3.

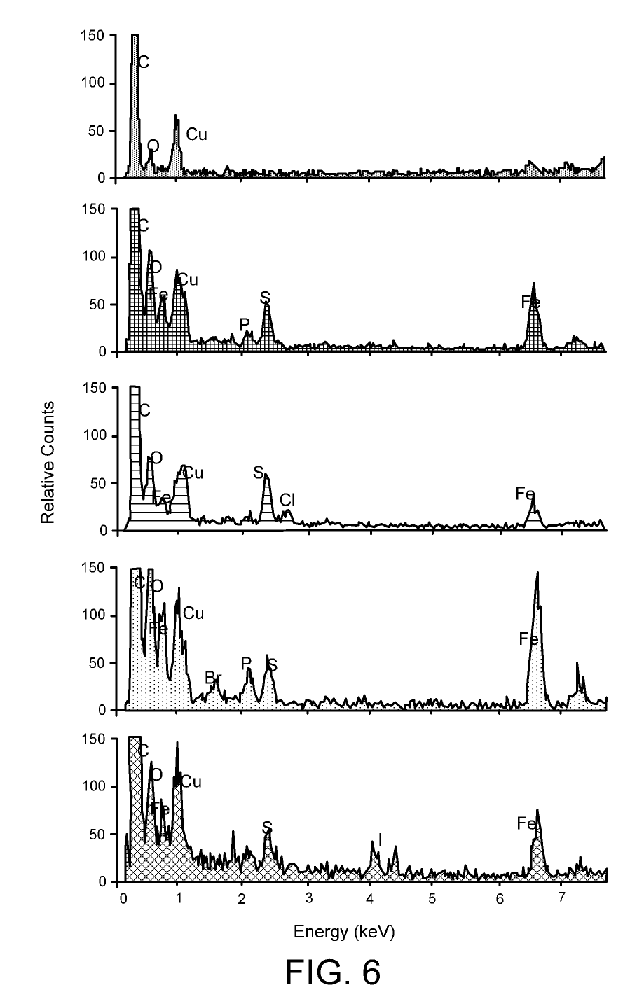

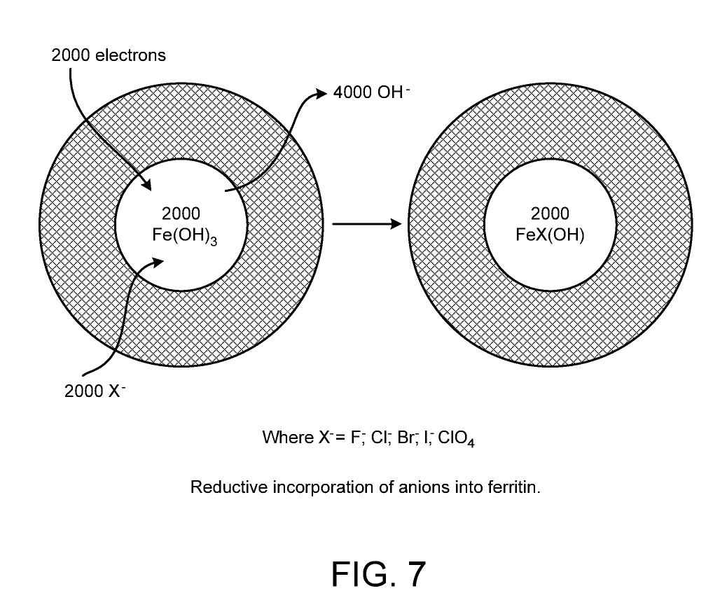

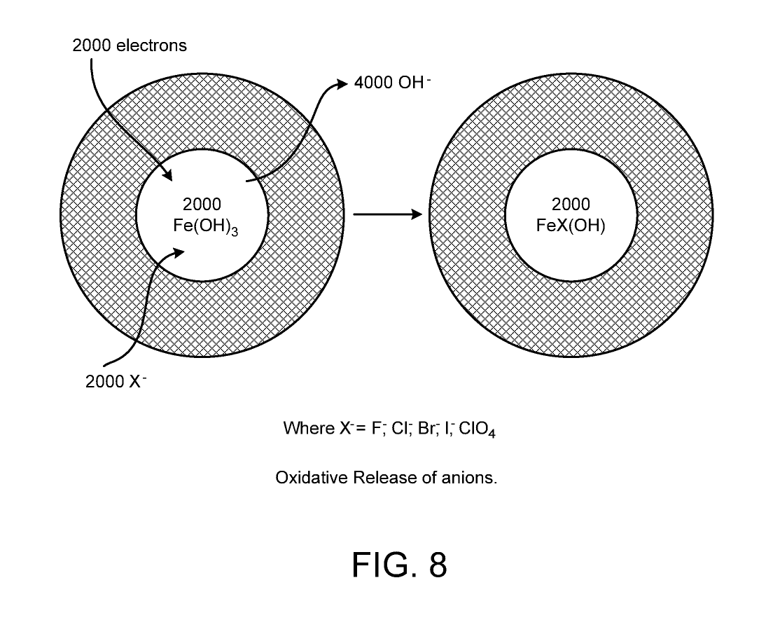

When the iron(III) core of ferritin is chemically reduced in the presence of iodide, the iodide anions are pumped inside the protein shell and removed from the exterior solution. The iodide ions that are pumped into the ferritin shell are no longer detected by the ISE. This indicates that the iodide has been pumped into the ferritin interior and is not accessible or free in solution to be measured by the ISE. Similar results have been obtained using fluoride, chloride, bromide and iodide as well as perchlorate (FIG. 6). Upon oxidation of the iron(II) core, the iodide or other anions are pumped back out of ferritin (FIG. 8). The anion pump of ferritin provides a way to sequester a large number of iodide anions inside a bio-compatible molecule and the release of these anions can be controlled by oxidation reactions.

Ferritin Nanocages

Ferritin protein nanocages are recognized for their ability to store metal ions. The metal sequestering ability of ferritin has been used for the size-constrained synthesis of many metal nanoparticles. In addition to metal cation sequestration, ferritin is required to sequester large quantities of anions to compensate for the charge of the metal ions. The counter anion for physiological mineral formation with iron is OH−, but the core also contains ˜10% phosphate in mammalian ferritin and significantly more phosphate if the ferritin comes from bacterial sources. Other oxoanions can substitute for OH− or phosphate during iron loading in ferritin. Most of the characterized reactions with anion loading into ferritin have been performed by adding the anion at the same time as the metal is deposited into ferritin. This co-deposition process leads to simultaneous deposition of the cation and anion. In its natural state, ferritin's core metal cation is iron but other embodiments of ferritin's metal core are known to include other divalent metal cations including cobalt, manganese, vanadium, nickel, zinc, copper, and silver.

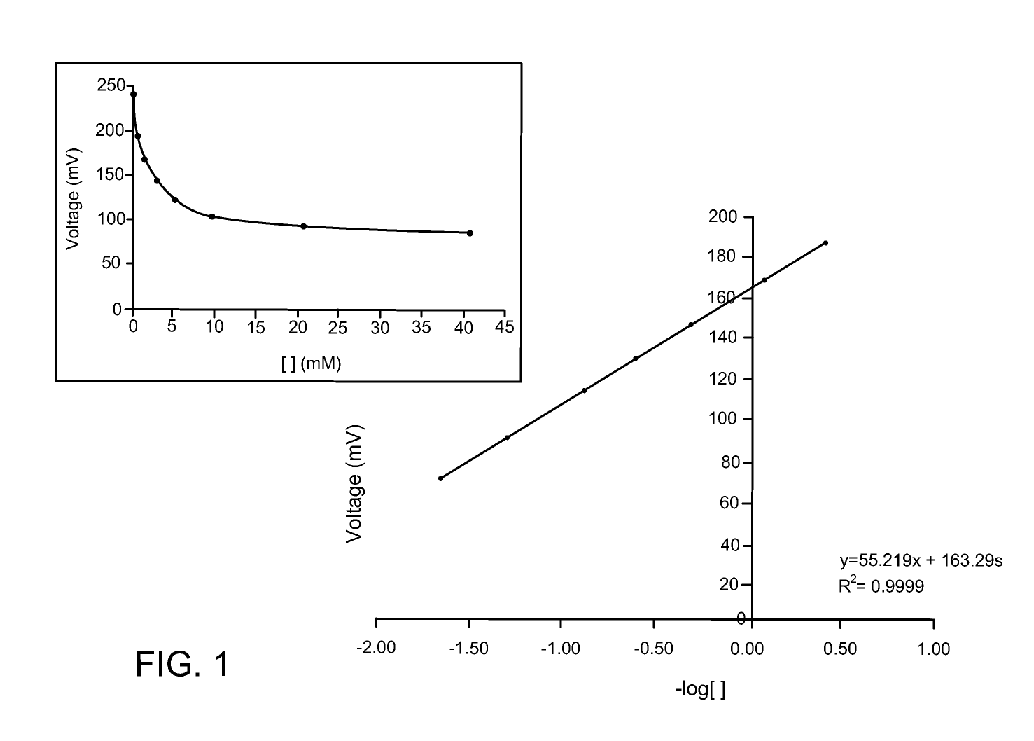

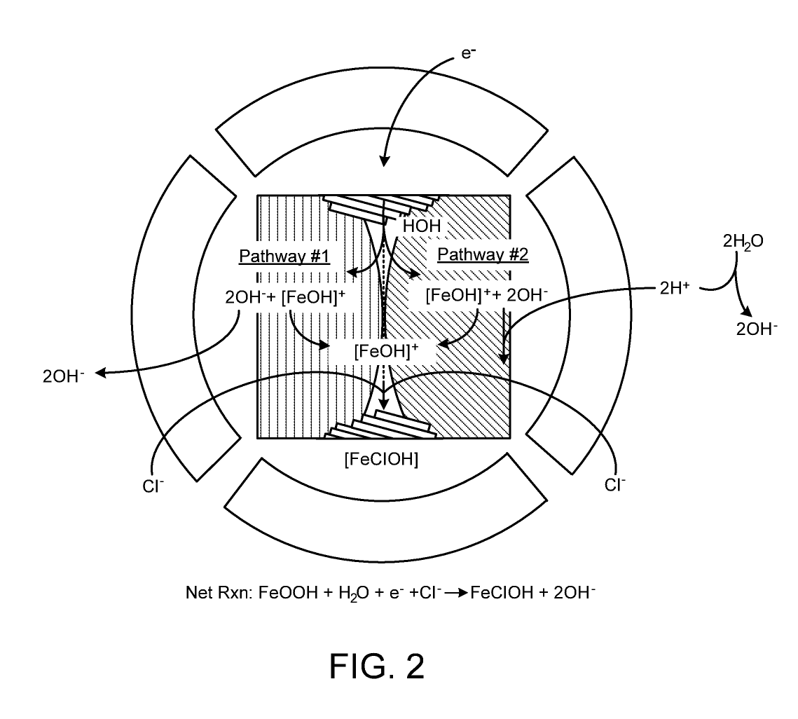

In contrast to iron loading, iron release occurs by a reductive process that requires Fe(II) chelators to remove iron from ferritin. Upon reduction of the FeOOH mineral core, the iron mineral remains safely sequestered inside ferritin unless an Fe(II) chelator is present to remove the iron. As part of the iron release mechanism the addition of electrons to reduce Fe(III) to Fe(II) is essential to make the iron more mobile. However, the addition of electrons disrupts the charge balance that gives the ferritin core stability. Reduction produces a charge imbalance that must be compensated for by the expulsion of anions. Studies monitoring pH changes that accompany chemical reduction of the ferritin core show that two OH− ions are released for each electron used to reduce Fe(III) to Fe(II) in the iron core to form the Fe(OH)+ intermediate. However, this equation fails to provide charge balance inside ferritin. Subsequent studies established that chloride present in the reaction buffer entered the ferritin interior to provide charge balance (Net Equation in FIG. 1) In the absence of halides in the buffer, only one hydroxide ion is expelled from ferritin, forming Fe(OH)2 as the mineral phase inside ferritin. The large number of iron atoms present in native ferritin (typically 1,500-2,500/ferritin) requires a large number of halides or hydroxides to cross the protein shell in response to redox changes in the iron mineral core (FIG. 2). The prevailing mechanism for iron loading suggests that Fe2+ binds to the ferritin protein and migrates to an enzyme site, named the ferroxidase center where oxidation to Fe3+ occurs. After oxidation, the iron migrates from the ferroxidase center to the protein interior where it forms the iron mineral.

Redox Loading of Ferritin

Provided herein are oxidation reduction reactions (Redox) that facilitate and substantially augment the loading of anions into the ferritin molecule. The eight ferritin channels at the 3-fold axes have shown the potential to transport of hydrophilic drugs, labeling fluorophores and potential nuclear medicine, metal ions such as cobalt, manganese, metallic phosphate, chromium, cadmium sulfide, iron sulfide, semiconductors (CdS), or some molecules in the size range of 3-4 A°. The remaining channels are hydrophobic, possibly facilitating transport of hydrophobic drugs or potential therapeutic agents such as imaging agents (Gd-HPDO3A). To this extent, redox reactions play a significant role in iron entry and release from ferritin. Several facets of this complex process have been actively investigated including the ferroxidase center, the nucleation sites, the redox properties of the mineral core, the redox reactivity of the associated phosphate layer, and the redox reactivity of the protein shell itself. However, processes associated with ion transfer across the protein shell, particularly anions required for charge balance, have been largely ignored. When iron is reduced in the mineral core, due to incoming electrons, a concomitant release of negative charge from the core must occur to balance the incoming negative charge. Experiments related to redox reactions that involve ion transfer across the protein shell of ferritin highlight this important process.

Anion sequestration during ferritin core reduction may be monitored using ion selective electrodes, elemental analysis, and energy-dispersive X-ray spectroscopy. When the core of equine spleen ferritin is fully reduced, a variety of anions, cross the protein shell and enter the ferritin interior. Reduction of the ferritin iron core initiates the release of two OH− ions per iron and sequesters one anion per iron inside ferritin in a charge balancing reaction. A general trend shows that smaller anions accumulate in greater abundance than larger anions, presumably because the protein channels restrict the transfer of the larger anionic species. Fe(II) remains stably sequestered inside ferritin as shown by electron micrograph imaging. Upon oxidation of the iron core, the halides are expelled from ferritin, returning the iron to the original Fe(O)OH mineral. The net overall reaction shows that halide ions accumulate inside ferritin upon chemical reduction of the core but upon oxidation of the iron core, halides are released from ferritin. The release and accumulation of hydroxide ions is opposite of halides. Hydroxide ions are released from ferritin upon iron reduction and accumulate in ferritin upon iron oxidation. Anion transport across the ferritin protein shell represents an important mechanistic function of ferritin. Herein provided is an anion exchange mechanism of ferritin representing a new method to synthesize a new class of materials inside ferritin using this newly elucidated reaction pathway.

In summary, redox events trigger the release of anions from ferritin, providing a redox-initiated anion release switch that is capable of changing the pH or ionic strength of solutions. In various embodiments ferritin may be loaded with a halide, a oxoanion, other anion, a radioisotope; and combinations thereof. In some embodiments a halide may be Fluoride (F−), Chloride (Cl−), Bromide (Br−), Iodide (I−), and combinations thereof. In other embodiments, this method can import an oxoanion class of compounds such Nitrate (NO3−), Rhenium Trioxide (ReO3−), Phosphate (PO43−), Vanadate (VO3−), Perchlorate (ClO4−), Molybdate (MoO43−), and Tungstate (WO43−) as well as other known anions including Cyanide (CN−), Thiocynate (SCN−), and Azide (N3−). These compounds have been shown to have therapeutic benefits at optimal dosages but show toxic effects when their dosage exceeds the optimal level. For example, thiocynate has been shown to be a potent vasodilator but toxicity develop with continued use.

In various embodiments, a buffered solution containing ferritin is combined with a solution containing the anion salt of the preselected anion, where the molar concentration of the iron from the ferritin solution equals to the molar concentration of the preselected anion. The combined solution is place in an deoxygenated environment and chemically reduced overnight with a reducing agent to completely reduce ferritin's iron core. The preferred reducing agent is formamidine sulfunic acid, which does not affect downstream reactions. The reducing agent is added in a 2:1 ratio of acid to ferritin concentration. The reduction of the metal core creates a charge imbalance that promotes the incorporation of the preselected anion.

### Therapeutic Use

Radioisotopes may be used to kill cancer cells. In certain embodiments various isotopes including but not limited to Molybdenum-99m (⁹⁹mMo); Technetium-99m (⁹⁹mTc); Lutetium-177 (¹⁷⁷Lu); yttrium-88 (⁸⁸Y); yttrium-90 (⁹⁰Y); Rhenium-186 (¹⁸⁶Rh); Rhenium 188 (¹⁸⁸Rh); Iodine 123 (¹²³I); Iodine 124 (¹²⁴I); Iodine-125 (¹²⁵I); Iodine-131 (¹³¹I); phosphorus-32 (³²P); phosphorus-33 (³³P); bromine-77 (⁷⁷Br); and combinations thereof are incorporated into ferritin nanocages. In some embodiments the ferritin is equine spleen holoferritin HoSF. In certain embodiments the ferritin nanocage comprises at least 1000 molecules of the passenger isotope. In some embodiments the ferritin nanocage comprises from 1000 to 1500, from 1500 to 2000, from 2000 to 2500, from 2500 to 3000, from 3000 to 3500 or from 3500 to 4000 molecules of passenger isotope.

The isotope loaded ferritin may be employed for various therapeutic uses, including cancer treatment. For non-limiting example, the radioisotope 1 is used to kill thyroid cancer cells because thyroid cells specifically incorporate iodide ions. Ferritin chemically reduced in the presence of 131-1 will allow incorporation of 131-1 into ferritin. An iron core of 1700 Fe/ferritin has incorporated 1350 iodide anions/ferritin using the process provided herein. This allows high concentrations of iodide ions to be sequestered inside a biocompatible delivery system.

In various embodiments isotope loaded ferritin may be targeted to treat cancers including but not limited to bladder cancer, lung cancer, bone cancer, breast cancer, brain cancer, colon and rectal cancer, endometrial cancer, kidney (renal cell) cancer, leukemia, non-Hodgkin lymphona, pancreatic cancer, and prostate cancer.

In certain embodiments chemically reduced ferritin containing 131-1 or other radioisotope is coated with antibodies to target the ferritin to bind specifically to the exterior surface of thyroid cancer cells. Because hemoglobin binds oxygen in the bloodstream, the free oxygen level is about 10 times lower than aerobic solutions. This means that the oxidation of the iron(II) in ferritin may be very slow and the release of 131-1 or other radioisotope may occur after the ferritin is attached to thyroid tumor cells. This targeting method may allow release of the radioisotope in the localized area of the thyroid cells for efficient incorporation into these cells. The targeting procedure and time release can make radioisotope delivery more efficient and require lower doses of radioactive material.

Ferritin has been shown to protect and stabilize radioactive ions and the enhanced loading amount of each nanoparticle in the process provided here is relatively even. Cancer treatments using these nanoparticles are highly successful due to the higher radiation doses from nanoparticles on cancer cells. 90Yttrium has also been used for experimental radioimmunotherapy against colorectal cancer, liver cancer, and ovarian cancer, among others. Lutetium-177 is a radionuclide of interest for radioimmunoimaging and radioimmunotherapy on account of its practical physical half-life (6.7 days) and its radiological properties, emitting both beta and gamma radiation. 177Lutetium appears feasible for many clinical applications including treatment of neuroendocrine tumors, carcinoid and pancreatic endocrine tumors, prostate, breast, and lung tumors, leukemias, and metastatic bone cancers. Their synthesis uses the hydrophilic channel to diffuse both radioactive and non-radioactive lutetium or yttrium into the apoferritin cavity. Research has also shown that ferritin has been used a vehicle to mediate anti-angiogenesis therapy. Also, the incorporation of various anticancer drugs in the ferritin core has shown reduced drug toxicities and drug resistance, while improving drug stability and efficacy in the delivery.

Commercial and Industrial Use

In some embodiments the process provided is used to electrochemically control the release or uptake of hydroxide ions from ferritin (PH pump system) as well as the pumping of anions in and out of ferritin depending on the redox status of the iron in the ferritin core. Thus, novel minerals may be formed in ferritin by this procedure for exchanging anions into and out of ferritin. For non-limiting example, iron perchlorate may be formed inside ferritin and in some embodiments produces a bio-nano-propellant capable of use as a rocket fuel. In certain embodiments iron azide is formed as an explosive with potential use in air bag materials and other applications.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided. One skilled in the relevant art will recognize, however, that the invention can be practiced without one or more of the specific details, or with other methods, components, materials, and so forth. In other instances, structures, materials, or operations that are known in the art are not shown or described in detail to avoid obscuring aspects of the invention.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Formulation and Delivery

Ferritin nanocages may be formulated for delivery including with a pharmaceutically acceptable carrier, or compound. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, nasal, optical, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, other fluids configured to preserve the integrity of the liposome, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, and sodium chloride sometimes are included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above.

Systemic administration can also be by transmucosal or transdermal means, including nasal and optical. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Delivery vehicles can also be prepared in the form of suppositories (e.g., with conventional suppository bases such as cocoa butter and other glycerides) or retention enemas for rectal delivery.

In some embodiments parenteral compositions are formulated in a dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD.sub.50 (the dose lethal to 50% of the population) and the ED.sub.50 (the dose therapeutically effective in 50% of the population). The dose ratio between toxic and therapeutic effects is the therapeutic index and it can be expressed as the ratio LD.sub.50/ED.sub.50. Molecules which exhibit high therapeutic indices often are utilized. The composition provided herein enables molecules that exhibit toxic side effects to be used, by emplouing a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules, here in some embodiments loaded ferritin, often lies within a range of circulating concentrations that include the ED.sub.50 with little or no toxicity. The dosage may vary within this range depending upon the dosage form employed and the route of administration utilized. For molecules used in the methods described herein, the therapeutically effective dose can be estimated initially from cell culture assays. A dose may be formulated in animal models to achieve a tissue concentration range that includes the IC.sub.50 (i.e., the concentration of the test compound which achieves a half-maximal inhibition of symptoms or destruction of abnormal cells) as determined in cell culture. Such information can be used to more accurately determine useful doses in humans. In vitro levels may be measured, for example, by high performance liquid chromatography. Another example of effective dose determination for an individual is the ability to directly assay levels of “free” and “tissue bound” compound in the serum of the test subject. Such assays may utilize antibody mimics and/or biosensors. Antibody conjugates may be used for targeting the composition disclosed herein to specific target cells. biological activity.

In some embodiments, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated, particularly when one delivers the molecule directly to the target cell. When one or more of these small molecules is to be administered to an animal (e.g., a human) in order to modulate expression or activity of a disease state, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Pharmaceutical compositions can be included in a container, pack, or dispenser together with instructions for administration. Pharmaceutical compositions of active ingredients can be administered by any of the paths described herein for therapeutic and prophylactic methods for treatment. With regard to both prophylactic and therapeutic methods of treatment, such treatments may be specifically tailored or modified, based on knowledge obtained from pharmacogenomic analyses described herein.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided. One skilled in the relevant art will recognize, however, that the invention can be practiced without one or more of the specific details, or with other methods, components, materials, and so forth. In other instances, structures, materials, or operations that are known in the art are not shown or described in detail to avoid obscuring aspects of the invention.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

### EXAMPLES

### Example 1

### Materials and Methods

Equine spleen ferritin (HoSF) was purchased from Sigma. The solution was dialyzed into buffer (0.025 M MOPS, pH 7.4) to eliminate the 0.15 M NaCl present in the stock buffer. The dialysis allowed the preparation of a defined amount and type of halide present in solution. The iron content of ferritin was 1,500 Fe/ferritin, as determined by reducing the iron using sodium hydrosulfite followed by chelation with 2,2′-bipyridine (bipy). The Fe-bipy complex has peak maxima at 520 nm, with an extinction coefficient of 8,400 M−1 cm−1. Protein concentrations were determined using the Lowry protein method. Anion salts, including NaF, NaCl, NaBr, NaI, NaClO4, Na2MoO4, NaH2PO4, NaReO4, NaNO3, NaVO3, Na2WO4, were obtained from Fisher Scientific or Sigma-Aldrich, and stock solutions of 1.00 M were prepared. Formamidine sulfinic acid (FSA) was obtained from Sigma, and fresh stock solutions of 0.1 M were prepared anaerobically immediately prior to use. Ferritin was combined with an equimolar amount of anion (˜1 anion per Fe atom) and then de-oxygenated and placed in an oxygen free glove box. FSA was added to the ferritin samples in an excess amount (2 FSA:1 Fe) to ensure complete reduction of the entire iron core. The reaction was allowed to stir over night.

Ion Selective Electrodes. Ion selective electrodes (ISEs) specific for several anions were purchased from Oakton Instruments, Vernon Hills, Ill. A standard curve was generated directly prior to use (FIG. 1), and the concentration of anion prior to reduction was determined. The concentration of anion following reduction by FSA was then determined. In this case, the concentration of anion as measured using the ISE corresponds to the concentration of anion that is free in solution, or anion that is not sequestered within the interior of ferritin. Thus, the concentration inside ferritin was determined by subtracting the final concentration from the initial concentration. The samples were then exposed to oxygen, and the concentration of anion was again determined. The re-oxidized samples had anion concentrations identical to the starting concentrations. Controls were set up to determine whether FSA interfered with the ISEs readings. FSA was selected as the reductant because unlike other reductants tested, FSA did not interfere with the ISE.

### Example 2

### Inductively Couple Plasma Emission (ICP)

ISEs were not available for purchase for every type of anion tested. In these cases, the samples were analyzed by inductively coupled plasma spectroscopy (ICP) on a PerkinElmer Optima 2000 DV. In this case, it was necessary to run the reduced samples over a column to separate the anions sequestered within ferritin from the excess anions free in solution. To do this, a GE-Healthcare PD-10 Sephadex G-25 column that was packed and placed in an oxygen free glove box. De-aerated buffer was passed over the column prior to the addition of the anaerobic reduced sample. The chromatography and fractionation was all performed in the oxygen free environment of the glove box. One mL fractions were collected, and each fraction was tested for protein content using the BCA method (FSA interferes with the Lowry method), and also for elemental content using ICP. All samples were compared against a standard curve. Appropriate controls were treated identically.

### Example 3

### UV-Vis Spectrophotometry

A spectrophotometer was placed in an oxygen free glove box. Samples were prepared as above, and the spectra prior to reduction and following reduction were determined. Controls without ferritin were also collected to show that the FSA peak is not responsible for the significant peaks shown (FIG. 6).

### Example 4

### Electron Microscopy (EM)

EM samples were prepared in the glove box to eliminate the opportunity for oxidization in the solution prior to placement on a grid. Grids were prepared by placing 4 μL of sample on a charged lacey carbon film, 400 mesh copper grids (Ted Pella, Inc.). Following 30 to 60 seconds on the grid, the solution was wicked off the grid, and the grid was rinsed in water to remove any salts or buffers. The grids were not stained in order to avoid contamination from the uranyl acetate. The grid was allowed to dry prior to removal from the glove box. Control samples were also prepared that contained no ferritin in order to determine the degree of unspecific binding by salts and buffers. The grids were then analyzed using a Tecnai F30 TEM, 140 kV. Energy dispersive x-ray spectroscopy (EDX) was used on the various samples (FIG. 6).

### Example 5

### Results

To determine the extent that chloride could enter the ferritin interior and to determine if other halides or anions could substitute for chloride, ISEs for the respective anions were used to monitor anion accumulation inside the ferritin nanocage in response to reduction of the ferritin iron core. Ferritin containing 1,500 Fe/ferritin (5 mM total iron in the reaction solution) was incubated with a 5 mM concentration of the indicated anion. When the anions were transported across the ferritin protein shell and sequestered inside ferritin, they could not be detected by the ISE. The results of these reactions are summarized in Table 1. The general trend shows that smaller anions traverse the ferritin shell more efficiently than the larger anions. Control reactions using apoferritin showed that anions were unable to accumulate inside ferritin in the absence of the iron mineral. In addition, ferrihydrite nanoparticles that are 8 nm in diameter, but lack a protein shell, were subjected to the same reductive conditions in the presence of anions. No change was detected in anion concentration using these ferrihydrite nanoparticle controls. Thus, neither the protein alone nor the ferrihydrite mineral alone could account for the change in anion concentration, supporting the model that the iron mineral inside of ferritin must be reduced, at which time the protein shell was able to sequester anionic species.

The pathway for anion influx into ferritin is unknown, but the most likely route is through channels that penetrate the ferritin nanocage at the 3-fold or 4-fold symmetry axes. However, the properties of these channels are not favorable for anion migration into the interior. The 3-fold channels are negatively charged and hydrophilic and the 4-fold channels are hydrophobic. In addition, both channels have a 0.4 nm diameter (200 pm radius), which is the approximate size of, if not smaller than, most of the anions, even without taking into account the size of the anions when hydrated (Table 1). Based on the properties of these channels it is remarkable that anions, especially the larger anions, are capable of entering the ferritin interior. These anions may require breathing modes of the protein that increase the size of the pores for anion entry into ferritin. The decreased loading of ferritin with larger halides is consistent with size and charge repulsion slowing the entry of these halides.

TABLE 1
ISE and ICP determinations of anion transfer into ferritin.

| | Ionic radius, | % of anion that | Anions atoms that | Fe/anion inside | [Anion] inside |
|---|---|---|---|---|---|
| Anion | pm | enters ferritin | enter ferritin | ferritin | ferritin, M |
| F⁻ | 136 | 100 ± 12 | 1,585 ± 165 | 1.0 ± 0.1 | 9.8 ± 1.0 |
| NO₃⁻ | 145 | 26.2 ± 10.0 | 393 ± 151 | 3.81 ± 0.35 | 3.91 ± 0.53 |
| Cl⁻ | 181 | 94.2 ± 7.8 | 1,278 ± 114 | 1.17 ± 0.08 | 7.92 ± 0.71 |
| ReO₄⁻ | 188 | | | | |
| Br⁻ | 195 | 72.9 ± 2.9 | 1,078 ± 84 | 1.39 ± 0.04 | 6.68 ± 0.52 |
| PO₄³⁻ | 210 | 12.1 ± 4.4 | 181 ± 124 | 1.32 ± 0.23* | 1.12 ± 0.63 |
| I⁻ | 216 | 80.7 ± 2.3 | 1,210 ± 53 | 1.24 ± 0.04 | 7.49 ± 0.33 |
| ClO₄⁻ | 220 | 57.6 ± 6.6 | 854 ± 303 | 1.76 ± 0.20 | 5.29 ± 1.88 |
| VO₄³⁻ | 258 | 79.4 ± 5.5 | 1,190 ± 108 | 1.26 ± 0.16 | 7.38 ± 0.81 |
| MoO₄³⁻ | 264 | 92.0 ± 4.5 | 1,380 ± 158 | 1.09 ± 0.24 | 8.55 ± 0.94 |
| WO₄³⁻ | 268 | 14.5 ± 2.3 | 217 ± 22 | 6.91 ± 0.28 | 1.34 ± 0.46 |

[ferritin] = 1.5 mg/ml, [Fe] = 5 mM, Fe/ferritin = 1,500, initial [anion] in solution = 5 mM, [FSA] = 10 mM.
*The phosphate sample chelated iron from ferritin. Thus, the Fe:anion ratio appears close to one as a result of very little iron remaining inside of ferritin. In other samples, the iron content remained very close to the original amount.

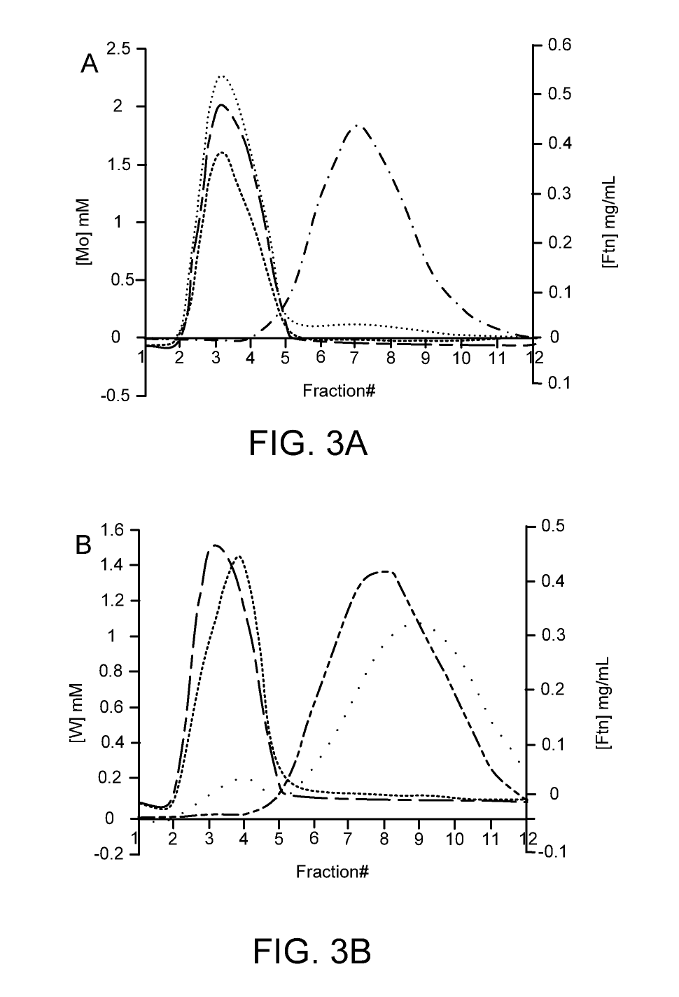

The reactions were all initiated with a 1:1 iron to anion ratio. In the case of the halides, following reduction of the iron core, essentially all of the halide ions present in solution entered the ferritin interior. The 1:1 association of halides with iron is evidence that upon reduction, the iron core must change properties from a crystalline mineral to an amorphous, porous state so that the halides can intercalate in the mineral phase and interact with each individual iron atom. This reduced ferritin mineral has not yet been characterized but should provide a very interesting mineral phase to study. The percentage of oxoanion incorporation into the ferritin core is not as high as that for the halides. Nevertheless, it represents the greatest number of oxoanion incorporation into ferritin that we are aware of in the current literature. The significant input of oxoanion into ferritin provides strong support for the effectiveness of this method for creating novel anion-iron minerals within ferritin. Note: for anions that were determined using ICP, a separation technique was required wherein the samples were run anaerobically over a size exclusion column and collected the fractions. Each fraction was analyzed for protein, iron, and the element of interest (such as Mo, V, and etc.) using techniques described in Materials and Methods. Representative elution profiles are shown in FIG. 3. The MoO42− and WO4− profiles are shown in 2A and 2B, respectively. A nearly 100% incorporation of MoO42− into the ferritin interior was observed, as evidence by the peak shift from fraction 10 to fraction 4, which contains the ferritin peak. However, WO4− only incorporates about 15% into ferritin, as shown by the minimal peak shift, and the remaining 85% remains soluble outside of ferritin.

### Example 6

### Phosphate Incorporation

Phosphate could be incorporated into ferritin in a nearly 1:1 ratio of phosphate to iron, with iron cores up to about 1,000 iron atoms per ferritin. This represented a significant number of phosphate molecules are incorporated into ferritin. In contrast to phosphate incorporation in the presence of iron, using this reductive method, very little phosphate was incorporated into ferritin. This occurred because phosphate is able to chelate Fe(II) and upon reduction of the iron mineral core, phosphate is able to pulls Fe(II) out of ferritin. Phosphate is present in cells at a concentration of 10 mM, which suggests that reduction of the iron core will result in the removal of iron from ferritin under in vivo phosphate concentrations. This finding was confirmed when the sample supernatant was purified in the absence of oxygen by running it over an oxygen free G-25 column and collecting fractions. The fractions were analyzed for protein, iron, and phosphate content. The protein eluted in the same fraction as normal, but both iron and phosphate were completely absent from the sample. Iron and phosphate were observed to elute together in a later fraction. However, most of the iron and phosphate were found in the insoluble material that formed in the process of this reaction. Thus, the ability to form novel materials inside ferritin using this reductive anion incorporation technique only occurred when the anion of interest did not have iron chelating ability. Similar results were obtained when CN− was used. Instead of CN− entering ferritin, Fe(II) was chelated from ferritin and Fe(CN)64− was formed on the exterior of ferritin (data not shown).

### Example 7

### Concentration Calculations

The molar concentration of the anion inside ferritin was calculated using the moles of halide and dividing by the calculated internal volume of ferritin. Ferritin, with an internal diameter of ˜8 nm, has an interior volume of 2.68×10-19 mL. The halide concentration approached 10 M for fluoride ion incorporation inside ferritin, which was a 2,000-fold increase over the original 5 mM fluoride concentration in solution. Because the movement of the anions was against a concentration gradient, the driving force for the reaction must come from the charge imbalance that occurs when two OH− ions were expelled from the ferritin interior for each Fe(III) reduced in the iron core (Eq. 1 and Eq. 2).

Interestingly, upon oxidation, the reverse reaction occurred and anions were expelled from ferritin. OH− ions either traverse the ferritin protein shell to return to the interior, or water present on the inside of ferritin is hydrolyzed to form the iron oxyhydroxy mineral core. The exact mechanism of OH− ion movement or proton movement across the protein shell has not yet been established, but the net yield is two OH− ions traverse the shell in response to reduction or oxidation of the iron core (See FIG. 2).

### Example 8

### Additional Studies

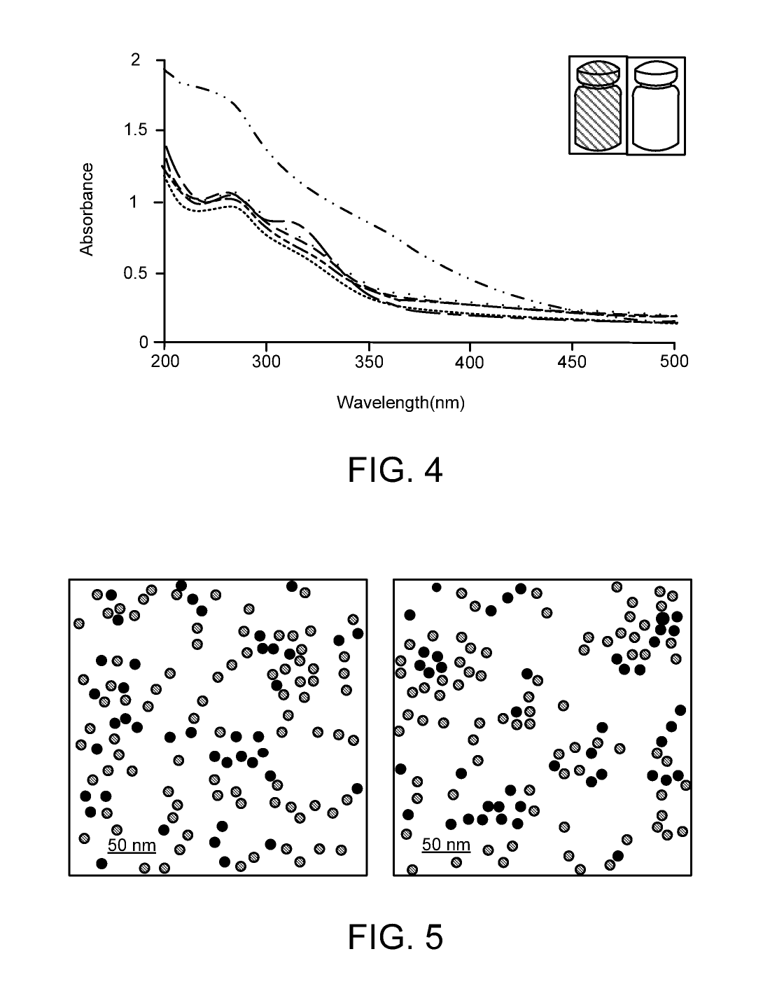

Several additional studies were used to confirm anion entry into ferritin. Native ferritin possesses a characteristic spectrophotometric signature. The peak at 280 nm represents the protein, and the broad shoulder tailing into the visible region (from 300-500 nm) represents the Fe(O)OH mineral sequestered inside ferritin. The oxidized ferritin solution appears reddish-brown (FIG. 3, left inset). Upon reduction, the reddish-brown color is replaced with a very faint yellow to colorless appearance (FIG. 3, right inset), and the broad visible shoulder from 300-500 nm decreases significantly (This is true in most cases. One exception is the formation of the iron-molybdate core, where the solution goes from reddish-brown to greenish-brown.). Reduction of ferritin in the presence of anion produces a similar spectrum to reduced ferritin but with slight variations, particularly around 320 nm, due to anion interactions with Fe(II). Reduction of ferritin in the presence of chloride shows the most significant spectral changes around 320 nm, compared to the other halides (FIG. 4). The slight spectral variation around 320 nm when different halides are present supports the view that upon reduction, the halides accumulate inside ferritin and interact with Fe(II). Upon oxidation, the solution returns to the original reddish-brown color, and the spectrum returns to the oxidized ferritin spectrum. ISE data confirm that upon oxidation, the anions are expelled from ferritin to form the native Fe(O)OH mineral.

To further verify that the anions were sequestered inside the ferritin protein shell, ferritin samples were reduced in the presence of anions in an anaerobic glove box, placed on electron microscope grids, and washed extensively followed by EM analysis. FIG. 5 shows a representative EM image of ferritin before and after being reduced in the presence of anion. FIG. 4 also shows that Fe(II) remains sequestered inside the 8 nm interior of ferritin upon reduction.

In addition, the halide samples were studied using energy-dispersive x-ray spectroscopy (EDX) to determine the elemental composition inside ferritin (FIG. 5). The samples were prepared as described above in an anaerobic glove box. Identical washing procedures were used for ferritin samples and control samples of apoferritin and no ferritin. The scanning area was half a micron squared. This area samples an average of 1,500 ferritin molecules. The spectra show the expected peaks corresponding to each individual halogen element, except for F, because the Kα peak of F (0.677 keV) is buried in the La peak of Fe (0.705 keV). In all other spectra, each respective halogen peak is identifiable. Controls with apoferritin reduced in the presence of halides did not show halogen peaks with EDX analysis, nor did controls coating grids with the holoferritin and the individual halides without reduction where the identical washing procedure was performed prior to EDX analysis. To confirm the presence of these halides within the protein shell, STEM was used to selectively scan a ˜12 nanometer square region that contained a single ferritin molecule. The combined data support the claim that the respective halides accumulate inside the nanocage of ferritin.

### Example 9

### Suggested Mechanisms

If ferritin expelled only one OH− ion/iron it would remain charge balanced, yet ferritin expels two OH− ions/iron upon reduction and produces a state that requires anion import to maintain charge balance. A potential mechanism suggests that the expulsion of two OH− ions/iron may have evolved as a protective mechanism to prevent protein denaturation from elevated pH.

The reactions driving OH− ion efflux from ferritin may be better understood by evaluating the concentrations of species that form inside ferritin. Upon reduction of the iron, halides accumulate inside ferritin at concentrations approaching 10 M (Table 6-1), which is equal to the iron concentration inside ferritin. Even higher concentrations of OH− ions are liberated by the reduction of the ferritin iron core. The reduction of the FeOOH mineral of ferritin produces Fe(OH)+ and releases two OH− ions into the ferritin interior (Eq. 1). If these reactions happened instantaneously, the resulting OH− concentration inside ferritin would approach ˜20 M. Ferritin is stable up to ˜pH 10 but the concentration of OH− ions inside ferritin under these conditions would be ˜pH 15, which would denature the protein. To maintain protein structural integrity, ferritin must rapidly expel the OH− ions. If all of the OH− ions were released simultaneously, the concentration gradient driving this reaction would be powerful with an internal OH− ion concentration of ˜20 M and an external OH− ion concentration of 10-7 M at pH 7.0. To avoid OH− denaturation, ferritin must efficiently efflux OH− ions to maintain protein integrity. The elimination of only one OH− ion/iron would be insufficient to protect the protein from pH denaturation.

In a simplistic stepwise view, after reduction of the iron core, ferritin expels all of the free OH− ions in an attempt to lower the OH− ion concentration to a safe level and in the process becomes charge imbalanced. Ferritin now requires anions to compensate for the positive charge build-up inside ferritin. The available anions for charge balance are halides (the 5 mM concentration was chosen to mimic the in vivo chloride concentration) and OH− ions, present at 10-7 M at pH 7.0. Because the halides are present at a 50,000 times greater concentration, halides enter ferritin to balance the charge. In reality, such a stepwise reaction cannot occur, thus the efflux of OH− ions must be simultaneously matched by an influx of halide ions. Understanding these reactions and taking into account the properties of ferritin will allow ferritin to be used as a redox-switch nanocage actuator for the storage and redox-controlled release of OH− and halide ions.

Finally, these findings may have in vivo implications. In healthy humans, the average chloride concentration within a cell is ˜5 mM. The inside of a cell is a reducing environment, and iron release from ferritin is proposed to occur by a reductive mechanism. Because iron chlorides are more soluble than iron hydroxides, the accumulation of chloride ions inside ferritin upon reduction of the iron may be a specific biological mechanism to facilitate the solubility and release of iron from ferritin. This is in contrast to the view that ferritin would need to be degraded in the lysosome in order to release iron. The association of iron with chloride inside ferritin is an attractive model for iron release from ferritin that allows ferritin to remain available for future iron binding.

What is claimed is:

1. A process for production of a metal nanoparticle, which process comprises:
a. providing a first solution containing a protein nanocage complex comprising a hydrophobic metal core and an ion-transport mechanism;
b. providing a second solution containing a preselected anionic agent;
c. combining the first and second solutions into a third combined solution; and
d. applying an external method to the third combined solution to manipulate the metal core's redox state; wherein reduction of the metal core causes the preselected anionic agent to be imported and incorporated into the metal core.

2. The process of claim 1, wherein said first solution is a pH-adjusted MOPS buffer.

3. The process of claim 1, wherein said protein nanocage complex is selected from ferritin and ferritin-like protein complexes, which use the ion-transport mechanism to incorporate the preselected anionic agent into the metal core.

4. The process of claim 3, wherein the ferritin protein is an equine spleen holoferritin HoSF.

5. The process of claim 4, wherein the holoferritin protein contains from at least 1000 iron atoms per holoferritin.

6. The process of claim 1, wherein said hydrophobic metal core is comprises a transition metal.

7. The process of claim 6, wherein the transition metal is selected from the group consisting of cobalt, iron, manganese, vanadium, nickel, zinc, copper, and silver.

8. The process of claim 1, wherein said preselected anionic agent is selected from the group of anions consisting of: a halide; a oxoanion; other anion; a radioisotope; and combinations thereof.

9. The process of claim 8, wherein the second solution comprises an anion salt of the preselected anion agent.

10. The process of claim 8, wherein said halide is selected from a group consisting of: Fluoride (F−); Chloride (Cl−); Bromide (Br−); Iodide (I−); and combinations thereof.

11. The process of claim 8, wherein said oxoanion is selected from a group comprising: Nitrate (NO3−); Rhenium Trioxide (ReO3−); Phosphate (PO43−); Vandate (VO3−); Perchlorate (ClO4−); Molybdate (MoO43−); Tungstate (WO43−), and combinations thereof.

12. The process of claim 8, wherein said other anion is selected from the group consisting of: Cyanide (CN−); Thiocynate (SCN−); Azide (N3−); and combinations thereof.

13. The process of claim 8, wherein said radioisotope is selected from the group consisting of: Molybdenum-99m (99 mMo); Technetium-99m (99mTc); Lutetium-177 (177Lu); yttrium-88 (88Y); yttrium-90 (90Y); Rhenium-186 (186Rh); Rhenium-188 (188Rh); Iodine-123 (123I); Iodine-124 (124I); Iodine-125 (125I); Iodine-131 (131I); phosphorus-32 (32P); phosphorus-33 (33P); bromine-77 (77Br); and combinations thereof.

14. The process of claim 1, wherein said third solution comprises an equimolar combination of the preselected anionic agent and the metal core.

15. The process of claim 14, wherein the third solution is de-oxygenated and completely reduced by a reducing agent.

16. The process of claim 1, wherein the external method is a reducing agent that creates a charge imbalance within the core and induces the importation and incorporation of the preselected anionic agent into the metal core.

17. The process of claim 16, wherein said reducing agent is formamidine sulfinic acid

18. A composition comprising:
a. a ferritin protein nanocage;
b. at least 1000 molecules of a preselected anionic agent; and
c. an antibody targeted to a specific cell.

19. The composition of claim 18 further comprising a pharmaceutical formulation.

20. The composition of claim 18 wherein the preselected anionic agent is a radioisotope.

21. The composition of claim 18, wherein the radioisotope is Iodine-131.

22. The composition of claim 18, wherein the ferritin protein is an equine spleen holoferritin HoSF.

23. The composition of claim 22, wherein the antibody is targeted to a thyroid cell.

24. A method for at least one of diagnosing and treating a disease, comprising providing to a patient in need of treatment a composition comprising a ferritin nanocage, at least 1300 molecules of a radioisotope, and an antibody targeted to a specific cell.

25. The method of claim 23 wherein the patient is selected from the group consisting of mammal, bird, reptile, and amphibian.

26. The method of claim 25 wherein the disease is cancer.

27. The method of claim 26 wherein the disease is cancer.

28. The method of claim 27 wherein the mammal is a human.

29. The method of claim 28 wherein the radioisotope is Iodine-131.

30. The method of claim 29, wherein the disease is thyroid cancer.

31. A composition comprising:
a. a ferritin protein nanocage;
b. at least 1000 molecules of a preselected anionic agent;
c. at least 1000 molecules of a preselected cations within a metal core

32. The composition of claim 31 further comprising a bio-nano-propellant.

33. The composition of claim of 32, wherein the bio-nano-propellant is a rocket fuel.

34. The composition of claim of 31, further comprising an explosive.

35. The composition of claim 31, where in the preselected anionic agent is Perchlorate (ClO4−).

36. The composition of claim 31, where in the preselected anionic agent is Azide (N3−).

37. The composition of claim 31, where in the preselected anionic agent is Nitrate (NO3−).

38. The composition of claim 31, where in the preselected cation is a divalent cation.

39. The composition of claim 38, where in the divalent cation is Iron.
